(19) United States
(12) Patent Application Publication
(10) Pub. No.: US 2004/0063660 A1
(43) Pub. Date: Apr. 1, 2004

(54) Diasteriomers of S-adenosyl-l-methionine and uses thereof

(76) Inventor: Rolland F. Hebert, Seattle, WA (US)

Correspondence Address: ROLLAND HEBERT, 427 BELLEVUE AVE E. SUITE 301, SEATTLE, WA, 98102, US

(21) Appl. No.: 10/663,943

(22) Filed: Sep. 16, 2003

Related U.S. Application Data
(62) Division of application No. 09/943,243, filed on Aug. 30, 2001.
(60) Provisional application No. 60/229,151, filed on Aug. 30, 2000.

(51) Int. Cl. A61K 31/7076

(52) U.S. Cl. 514/46

(57) ABSTRACT

Methods of use of administration of substantially optically pure diasteriomeric forms of S-adenosyl-l-methionine salts or defined non-racemic ratios of (S,S)-S-adenosyl-l-methionine to (R,S)-S-adenosyl-l-methionine and their salts in treating various conditions are described.

### BACKGROUND-CROSS-REFERENCES TO RELATED APPLICATION

This is a divisional of U.S. patent application Ser. No. 09/943,243 filed on Aug. 30, 2001, now abandoned, the entire disclosure and contents of which are incorporated by reference.

### FIELD OF THE INVENTION

The present invention relates to methods of treating or preventing disease using substantially optically pure diasteriomers of S-adenosyl-l-methionine and defined non-racemic ratios of (S,S)-S-adenosyl-l-methionine to (R,S)-S-adenosyl-l-methionine.

### TECHNICAL FIELD

This patent relates to the use of substantially optically pure diasteriomers of S-adenosyl-l-methionine (SAM-e), defined non-racemic ratios of (S,S)-S-adenosyl-l-methionine to (R,S)-S-adenosyl-l-methionine. More particularly, the invention relates to the treatment and prevention of diseases and other conditions using substantially optically pure diasteriomer (S,S)-S-adenosyl-l-methionine, defined non-racemic ratios of (S,S)-S-adenosyl-l-methionine to (R,S)-S-adenosyl-l-methionine, pharmaceutically acceptable salts and pharmaceutical compositions that contain them as active principles.

### BACKGROUND OF THE INVENTION

Many organic compounds exist in optically active forms, i.e., they have the ability to rotate the plane of plane-polarized light. In describing an optically active compound, the prefixes D and L or R and S are used to denote the absolute configuration of the molecule about its chiral center. The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these compounds, called stereoisomers, are identical except that they are mirror images of one another. A specific stereoisomer may also be referred to as an enantiomer, and a mixture of such isomers is often called an enantiomeric mixture. A 50:50 mixture of enantiomers is referred to as a racemic mixture. A compound with more than one chiral center is a diasteriomer. S-adenosyl-l-methionine is a diasteriomer.

Stereochemical purity is of importance in the field of pharmaceuticals, where 12 of the 20 most prescribed drugs exhibit chirality. A case in point is provided by the L-form of the beta-adrenergic blocking agent, propranolol, which is known to be 100 times more potent than the D-enantiomer.

Furthermore, optical purity is important since certain isomers may actually be deleterious rather than simply inert. For example, it has been suggested that the D-enantiomer of thalidomide was a safe and effective sedative when prescribed for the control of morning sickness during pregnancy, and that the corresponding L-enantiomer was a potent teratogen. S-adenosyl-l-methionine is a naturally occurring substance that is present in all living organisms and has a number of very important biological functions. Among these functions are the following: methyl group donor in transmethylation reactions (it is the sole methyl group donor in such reactions-including methylation of DNA, proteins, hormones, catechol and indoleamines and phosphatidylethanolamine to phosphatidylcholine); it is a substrate of an enzyme lyase that converts S-adenosyl-l-methionine to the molecule methylthioadenosine and homoserine; it is an aminobutyric chain donor to tRNA; it is an aminoacidic chain donor in the biosynthesis of biotin; S-adenosyl-l-methionine, after decarboxylation, is the donor of aminopropyl groups for the biosynthesis of neuroregulatory polyamines spermidine and spermine. (Zappia et al (1979), Biomedical and Pharmacologcial roles of Adenosylmethionine and the Central Nervous System, page 1, Pergamon Press. NY.)

S-adenosyl-l-methionine has been used clinically in the treatment of liver disease (Friedel H, Goa, K. L., and Benfield P., (1989), S-adenosyl-l-methionine: a review of its pharmacological properties and therapeutic potential in liver dysfunction and affective disorders in relation to its physiological role in cell metabolism. Drugs. 38, 389-416), arthritis (Di Padova C, (1987), S-adenosyl-l-methionine in the treatment of osteoarthritis: review of the clinical studies. Am J. Med. 83, (Suppl. 5), 6-65), and depression (Kagan, B, Sultzer D. L., Rosenlicht N and Gerner R. (1990), Oral S-adenosyl-l-methionine in depression: a randomized, double blind, placebo-controlled trial. Am. J. Psychiatry 147, 591-595.) Alzheimer's patients have reduced cerebral spinal fluid levels of S-adenosyl-l-methionine (Bottiglieri et al, (1990), Cerebrospinal fluid S-adenosyl-l-methionine in depression and dementia: effects of treatment with parenteral and oral S-adenosyl-l-methionine. J. Neurol. Neurosurg. Psychiatry 53, 1096-1098.) In a preliminary study, S-adenosyl-l-methionine was able to produce cognitive improvement in patients with Alzheimer's disease. (Bottiglieri et al (1994), The clinical potential of admetionine (S-adenosyl-l-methioinine) in neurological disorders. Drugs 48, 137-152.) S-adenosyl-l-methionine brain levels in patients with Alzheimer's disease are also severely decreased. (Morrison et al, (1996), Brain S-adenosyl-l-methionine levels are severely decreased in Alzheimer's disease, Journal of Neurochemistry, 67, 1328-1331.) Patients with Parkinson's disease have also been shown to have significantly decreased blood levels of S-adenosyl-l-methionine. (Cheng et al, (1997), Levels of L-methionine S-adenosyltransferase activity in erythrocytes and concentrations of S-adenosyl-l-methionine and S-adenosylhomocysteine in whole blood of patients with Parkinson's disease. Experimental Neurology 145, 580-585.)

S-adenosyl-l-methionine levels in patients treated with the antineoplastic drug methotrexate are reduced. Neurotoxicity associated with this drug may be attenuated by co-administration of S-adenosyl-l-methionine. (Bottiglieri et al (1994), The Clinical Potential of Ademetionine (S-adenosyl-l-methionine) in neurological disorders, Drugs, 48 (2), 137-152.) Cerebral spinal fluid levels of S-adenosyl-l-methionine have been investigated in HIV AIDS dementia Complex/HIV encephalopathy and found to be significantly lower than in non-HIV infected patients. (Keating et al (1991), Evidence of brain methyltransferase inhibition and early brain involvement in HIV positive patients Lancet: 337:935-9.)

De La Cruz et al have shown that S-adenosyl-l-methionine, chronically administered, can modify the oxidative status in the brain by enhancing anti-oxidative defenses. (De La Cruz et al, (2000), Effects of chronic administration of S-adenosyl-l-methionine on brain oxidative stress in rats. Naunyn-Schmiedeberg's Archives Pharmacol 361: 47-52.) This is similar to results obtained with S-adenosyl-l-methionine in liver and kidney tissue. Thus S-adenosyl-l-methionine would be useful as an antioxidant.

Oral S-adenosyl-l-methionine administration to patients with and without liver disease has resulted in increases in liver glutathione levels. (Vendemiale G et al, (1989), Effect of oral S-adenosyl-l-methionine on hepatic glutathione in patients with liver disease. Scand J Gastroenterol; 24: 407-15. Oral administration of S-adenosyl-l-methionine to patients suffering from intrahepatic cholestasis had improvements in both the pruritus as well as the biochemical markers of cholestasis. (Giudici et al, The use of admethionine (S-adenosyl-l-methionine) in the treatment of cholestatic liver disorders. Meta-analysis of clinical trials. In: Mato et al editors. Methionine Metabolism: Molecular Mechanism and Clinical Implications. Madrid: CSIC Press; 1992 pp 67-79.) Oral S-adenosyl-l-methionine administration to patients suffering from primary fibromyalgia resulted in significant improvement after a short-term trial. (Tavoni et al, Evaluation of S-adenosylmethioine in Primary Fibromaylgia. The American Journal of Medicine, Vol 83 (suppl 5A), pp 107-110, 1987.) S-adenosyl-l-methionine has been used for the treatment of osteoarthritis as well. (Koenig B. A long-term (two years) clinical trial with S-adenosyl-l-methionine for the treatment of osteoarthritis. The American Journal of Medicine, Vol 83 (suppl 5A), Nov. 20, 1987 pp 89-94).

S-adenosyl-l-methionine is clinically useful in many apparently unrelated areas because of its important function in basic metabolic processes. One of its most striking clinical uses is in the treatment of alcoholic liver cirrhosis that, until now, remained medically untreatable. Mato et al demonstrated the ability of oral S-adenosyl-l-methionine in alcoholic liver cirrhosis to decrease the overall mortality and/or progression to liver transplant by 29% vs 12% as compared with a placebo treated group. (Mato et al (1999), S-adenosyl-l-methionine in alcohol liver cirrhosis: a randomized, placebo-controlled, double blind, multi-center clinical trial, Journal of Hepatology, 30, 1081-1089.)

S-adenosyl-l-methionine also attenuates the damage caused by tumor necrosis factor alpha and can also decrease the amount of tumor necrosis factor alpha secreted by cells. Consequently, conditions in which this particular inflammatory factor is elevated would benefit from the administration of S-adenosyl-l-methionine. (Watson W H, Zhao Y, Chawla R K, (1999) Biochem J August 15; 342 (Pt 1):21-5. S-adenosyl-l-methionine attenuates the lipopolysaccharide-induced expression of the gene for tumour necrosis factor alpha.) S-adenosyl-l-methionine has also been studied for its ability to reduce the toxicity associated with administration of cyclosporine A, a powerful immunosuppressor. (Galan A, et al, Cyclosporine A toxicity and effect of the S-adenosyl-l-methionine, Ars Pharmaceutica, 40:3; 151-163, 1999.)

S-adenosyl-l-methionine, incubated in vitro with human erythrocytes, penetrates the cell membrane and increases ATP within the cell thus restoring the cell shape. (Friedel et al, S-adenosyl-l-methionine: A review of its pharmacological properties and therapeutic potential in liver dysfunction and affective disorders in relation to its physiological role in cell metabolism, Drugs 38 (3):389416, 1989)

S-adenosyl-l-methionine has been studied in patients suffering from migraines and found to be of benefit. (Friedel et al, S-adenosyl-l-methionine: A review of its pharmacological properties and therapeutic potential in liver dysfunction and affective disorders in relation to its physiological role in cell metabolism, Drugs 38 (3): 389-416, 1989).

Belli et al in an article entitled “S-adenosyl-methionine prevents total parenteral nutrition-induced cholestasis in the rat”, Journal of Hepatology 1994; 21: 18-23 showed that S-adenosyl-l-methionine was able to prevent cholestasis resulting from total parenteral nutrition by maintaining liver plasma membrane enzymatic activity via preservation of the membrane lipid environment.

S-adenosyl-l-methionine has been administered to patients with peripheral occlusive arterial disease and was shown to reduce blood viscosity, chiefly via its effect on erythrocyte deformability.

Garcia P et al in “S-adenosylmetionine: A drug for the brain?”, IVth Workshop on Methionine Metabolism: Molecular Mechanisms and Clinical Implications”, Symposium held on March 1-5, Granada, Spain, 1998, reported that S-adenosyl-l-methionine was able to increase the number of muscarinic receptors in the brains of rats treated chronically with S-adenosyl-l-methionine. Muscarinic receptors in the brain, especially in the hippocampus, are important in learning and memory. In a standard eight arm radical maze test, treated animals were able to out-perform age matched older untreated animals. Young aged matched S-adenosyl-l-methionine treated animals were also able to out-perform young non-treated animals showing S-adenosyl-l-methionine's ability to increase memory even in young animals. The conclusions drawn were that S-adenosyl-l-methionine is able to improve memory not only in adult aged animals but also in young animals thus making S-adenosyl-l-methionine an eligible candidate therapy for the treatment of memory impairment and learning difficulties.

S-adenosyl-l-methionine is commercially available using fermentation technologies that result in S-adenosyl-l-methionine formulations varying between 60 and 80% purity. (That is, the final product contains 60-80% of the active or (S,S)-S-adenosyl-l-methionine and 20-40% of the inactive or (R,S)-S-adenosyl-l-methionine.) (Gross, A., Geresh, S., and Whitesides, G m (1983) Appl. Biochem. Biotech. 8, 415.) Enzymatic synthetic methodologies have been reported to yield the inactive isomer in concentrations exceeding 60%. (Matos, J R, Rauschel F M, Wong, C H. S-adenosyl-l-methionine: Studies on Chemical and Enzymatic Synthesis. Biotechnology and Applied Biochemistry 9, 39-52 (1987). Enantiomeric separation technologies have been reported to resolve the pure active diasteriomer of S-adenosyl-l-methionine. (Matos, J R, Rauschel F M, Wong, C H. S-adenosyl-l-methionine: Studies on Chemical and Enzymatic Synthesis. Biotechnology and Applied Biochemistry 9, 39-52 (1987; Hoffman, Chromatographic Analysis of the Chiral and Covalent Instability of S-adenosyl-l-methionine, Biochemistry 1986, 25 4444-4449: Segal D and Eichler D, The Specificity of Interaction between S-adenosyl-l-methionine and a nucleolar 2-0-methyltransferase, Archives of Biochemistry and Biophysics, Vol. 275, No. 2, December, pp. 334-343, 1989) Newer separation technologies exist to resolve enantiomers and diasteriomers on a large commercial production scale at a very economic cost. In addition, it would be conceivable to synthesize the biologically active diasteriomer using special sterioselective methodologies but this has not been accomplished to date.

De la Haba first showed that the sulfur is chiral and that only one of the two possible configurations was synthesized and used biologically. (De la Haba et al J. Am. Chem. Soc. 81, 3975-3980, 1959) Methylation of RNA and DNA is essential for normal cellular growth. This methylation is carried out using S-adenosyl-l-methionine as the sole or major methyl donor with the reaction being carried out by a methyltransferase enzyme. Segal and Eichler showed that the enzyme bound (S,S)-S-adenosyl-l-methionine 10 fold more tightly than the biologically inactive (R,S)-S-adenosyl-l-methionine thus demonstrating a novel binding stereospecificity at the sulfur chiral center. Other methyltransferases have been reported to bind (R,S)-S-adenosyl-l-methionine to the same extent as (S,S)-S-adenosyl-l-methionine and thus (R,S)-S-adenosyl-l-methionine could act as a competitive inhibitor of that enzyme. (Segal D and Eichler D, The Specificity of Interaction between S-adenosyl-l-methionine and a nucleolar 2-0-methyltransferase, Archives of Biochemistry and Biophysics, Vol. 275, No. 2, December, pp. 334-343, 1989; Borchardt RT and Wu YS, Potential inhibitors of S-adenosyl-l-methionine-dependent methyltransferases. Role of the Asymmetric Sulfonium Pole in the Enzymatic binding of S-adenosyl-l-methionine, Journal of Medicinal Chemistry, 1976, Vol 19, No. 9, 1099-1103.)

Borchardt and Wu, in an article entitled “Potential Inhibitors of S-adenosyl-l-methionine-dependent methyltransferases. 5. Role of the Asymmetric Sulfonium Pole in the Enzymatic Binding of Adenosyl-L-methionine”, Journal of Medicinal Chemistry, 1976, Vol. 19, No. 9, pp 1099-1103, report that the (+)-SAM (no longer used nomenclature for (R,S)-S-adenosyl-l-methionine) is a potent inhibitor of enzyme-catalyzed transmethylation reactions. Since transulferation and methylation reactions are the hallmark of S-adenosyl-l-methionine's mechanism of action, it would be prudent to use S-adenosyl-l-methionine with as enriched a concentration of (S,S)-S-adenosyl-l-methionine in any pharmaceutical composition as possible since the (R,S)-S-adenosyl-l-methionine diasteriomer may be inhibitory to the desired action of (S,S)-S-adenosyl-l-methionine.

Detich et al in an article entitled “The methyl donor S-adenosyl-l-methionine inhibits active demethylation of DNA; a candidate novel mechanism for the pharmacological effects of S-adenosyl-methionine.” J Biol Chem. Jun. 6, 2003;278(23):20812-20, point out the tumor protective mechanism of S-adenosyl-l-methionine and the importance of intracellular S-adenosyl-l-methionine concentrations in cancer prevention. Presumably this is due to the ability of S-adenosyl-l-methionine to prevent DNA hypomethylation. Indeed, DNA hypomethylation is a hallmark of cancer cells and the correction of this hypomethylation leads to proper gene expression and reversal or prevention of cancer. However, in light of the known inability of (R,S)-S-adenosyl-l-methionine to participate in methylation or transulfuration reactions (indeed, it inhibits these reactions), it becomes increasingly apparent that S-adenosyl-l-methionine compositions should contain the least amount of (R,S)-S-adenosyl-l-methionine possible. S-adenosyl-l-methionine (whether in its optically pure diasteriomeric form or in defined non-racemic ratios of (S,S)-S-adenosyl-l-methionine to (R,S)-S-adenosyl-l-methionine or as a racemic mixture) presents certain difficult problems in terms of its stability at ambient temperature that result in degradation of the molecule to undesirable degradation products as well as to epimerization to its less desirable form (R,S)-S-adenosyl-l-methionine. S-adenosyl-l-methionine (and thus its diasteriomers) must be further stabilized since they exhibit intramolecular instability that causes the destabilization and breakdown of the molecule at both high as well as ambient temperatures. S-adenosyl-l-methionine has therefore been the subject of many patents directed both towards obtaining new stable salts, and towards the provision of preparation processes that can be implemented on an industrial scale. The present patent thus envisions the use of any of the salts of S-adenosyl-l-methionine already disclosed in the prior art in order to stabilize the diasteriomeric forms of S-adenosyl-l-methionine. Examples of such salts disclosed in the prior art include, but not limited to, the following: a lipophilic salt of S-adenosyl-l-methionine of the formula S-adenosyl-l-methionine.sup.n+[R—CO—NH—(CH.sub.2).sub.2-SO.sup.-.sub.3].sub.n in which R—CO is a member selected from the group consisting of C.sub.12-C.sub.26 saturated and unsaturated, linear and branched acyl and C.sub.12-C.sub.26 cycloalkyl-substituted acyl, and n is an integer from 3 to 6 according to the S-adenosyl-l-methionine charge; double salts corresponding to the formula S-adenosyl-l-methionine.sup.+.HSO.sub.4.sup.-.H.sub.2 SO.sub.4.2 CH.sub.3 C.sub.6 H.sub.4 SO.sub.3 H.; salts (S,S)-s-adenosyl-l-methionine with sulphonic acids selected from the group consisting of methanesulphonic, ethanesulphonic, 1-n-dodecanesulphonic, 1-n-octadecanesulphonic, 2-chloroethanesulphonic, 2-bromoethanesulphonic, 2-hydroxyethanesulphonic, 3-hydroxypropanesulphonic, d-,1-,d,1-10-camphorsulphonic, d-,1,-d,1-3-bromocamphor-10-sulphonic, cysteic, benzenesulphonic,p-chlorobenzenesulphonic, 2-mesitylbenzenesulphonic, 4-biphenylsulphonic, 1-naphthalenesulphonic, 2-naphthalenesulphonic, 5-sulphosalicylic, p-acetylbenzenesulphonic, 1,2-ethanedisulphonic, methanesulphonic acid, ethanesulphonic acid, 1-n-dodecanesulphonic acid, 1-n-octadecanesulphonic acid, 2-chloroethanesulphonic acid, 2-bromoethanesulphonic acid, 2-hydroxyethanesulphonic acid, d-,1-,d,1-10-camphorsulphonic acid, d-,1-,d,1-3-bromocamphor-10-sulphonic acid, cysteic acid, benzenesulphonic acid, 3-hydroxypropanesulphonic acid, 2-mesitylbenzenesulphonic acid, p-chlorobenzenesulphonic acid,4-biphenylsulphonic acid, 2-naphthalenesulphonic acid, 5-sulphosalicylic acid, 1,2-ethanedisulphonic acid, p-acetylbenzenesulphonic acid, 1-naphthalenesulphonic acid, o-benzenedisulphonic and chondroitinesulphuric acids, and double salts of said acids with sulphuric acid; S-adenosyl-l-methionine or a pharmaceutically acceptable salt thereof and an effective amount of a lithium salt selected from the group consisting of lithium chloride, lithium bromide, lithium iodide, lithium sulfate, lithium nitrate, lithium phosphate, lithium borate, lithium carbonate, lithium formate, lithium acetate, lithium citrate, lithium succinate and lithium benzoate; water-soluble salt of a bivalent or trivalent metal is a member selected from the group consisting of calcium chloride, ferric chloride, magnesium chloride, and magnesium sulfate; the salt of S-adenosyl-l-methionine is a member selected from the group consisting of salts of S-adenosyl-l-methionine with hydrochloric acid, sulfuric acid, phosphoric acid, formic acid, acetic acid, citric acid, tartaric acid, and maleic acid; and a double salt of S-adenosyl-l-methionine with said acids; a salt of S-adenosyl-l-methionine and a water-soluble polyanionic substance selected from the group consisting of a polyphosphate, metaphosphate, polystyrene sulfonate, polyvinyl sulfonate, polyvinyl sulfate, polyvinyl phosphate, and polyacrylate wherein the stoichiometric ratio of mols of S-adenosyl-l-methionine to gram-equivalent of the polyanionic substance is from 0.1:1 to 0.5; a salt of S-adenosyl-l-methionine wherein the polyanionic substance is a polyphosphate, para-polystyrene sulfonate or metaphosphate; a salt of the general formula: S-adenosyl-l-methionine.nR(O).sub.m (SO.sub.3 H)p (I) where m can be zero or 1; n is 1.5 when p is 2, and is 3 when p is 1; R is chosen from the group consisting of alkyl, phenylalkyl and carboxyalkyl, in which the linear or branched alkyl chain contains from 8 to 18 carbon atoms, and in particular for producing S-adenosyl-l-methionine salts of sulphonic acids, or of sulphuric acid esters, or of dioctylsulphosuccinic acid. However the more preferred salts of the S-adenosyl-l-methionine diasteriomers are chosen from the group consisting of salts of S-adenosyl-l-methionine diasteriomers with sulfuric acid, p-toluenesulfonic acid, and 1,4-butanedisulphonic acid.

### PRIOR ART

Many patents exist disclosing salts of S-adenosyl-l-methionine that stabilize the molecule but none discloses the use of an optically pure diasteriomer of S-adenosyl-l-methionine or defined non-racemic ratios of (S,S)-S-adenosyl-l-methionine to (R,S)-S-adenosyl-l-methionine. U.S. Pat. No. 2,969,353, Shunk et al, Jan. 24, 1962, discloses a method for the preparation of S-adenosyl-l-methionine and a stable salt of S-adenosyl-l-methionine but not the use of an optically pure diasteriomer of S-adenosyl-l-methionine or defined non-racemic ratios of (S,S)-S-adenosyl-l-methionine to (R,S)-S-adenosyl-l-methionine. U.S. Pat. 3,707,536, Haid et al, Dec. 26, 1972, discloses a new S-adenosyl-l-methionine bisulfate salt but not the use of an optically pure diasteriomer of S-adenosyl-l-methionine or defined non-racemic ratios of (S,S)-S-adenosyl-l-methionine to (R,S)-S-adenosyl-l-methionine. U.S. Pat. No. 3,893,999, Fiecchi, Jul. 8, 1975, discloses a new salt of S-adenosyl-l-methionine made with tri-p-toluensulphonate but not the use of an optically pure diasteriomer of S-adenosyl-l-methionine or defined non-racemic ratios of (S,S)-S-adenosyl-l-methionine to (R,S)-S-adenosyl-l-methionine. U.S. Pat. No. 5,102,791, Gennari, Apr. 7, 1992, discloses, among others, a 1,4 butanedisulfonate salt of S-adenosyl-l-methionine but not the use of an optically pure diasteriomer of S-adenosyl-l-methionine or defined non-racemic ratios of (S,S)-S-adenosyl-l-methionine to (R,S)-S-adenosyl-l-methionine. U.S. Pat. No. 4,028,183, Fiecchi, Jun. 7, 1977, discloses, among others, p-toluene sulfonate as a means to stabilize the S-adenosyl-l-methionine molecule but does not disclose the use of an optically pure diasteriomer of S-adenosyl-l-methionine or defined non-racemic ratios of (S,S)-S-adenosyl-l-methionine to (R,S)-S-adenosyl-l-methionine. U.S. Pat. 4,764,603, Zappia, Aug. 16, 1988, discloses the use of polyanions such as polyphosphates, polyvinylsulfonates sulfates or phosphates, polyacrylates, and polystyrene sulfonates. However, this patent does not disclose the use of an optically pure diasteriomer of S-adenosyl-l-methionine.

U.S. Pat. 6,117,849, Zimmermann, et al. Sep. 12, 2000, discloses the use of S-adenosyl-l-methionine complexed with nucleosides as HIV inhibitors but does not disclose the use of an optically pure diasteriomer of S-adenosyl-l-methionine for any other condition nor a diasteriomer of S-adenosyl-l-methionine uncomplexed to another molecule nor defined non-racemic ratios of (S,S)-S-adenosyl-l-methionine to (R,S)-S-adenosyl-l-methionine. U.S. Pat. 4,465,672, Gennari, Aug. 14, 1984, discloses new S-adenosyl-l-methionine salts but does not disclose the use of an optically pure diasteriomer of S-adenosyl-l-methionine or defined non-racemic ratios of (S,S)-S-adenosyl-l-methionine to (R,S)-S-adenosyl-l-methionine. U.S. Pat. No. No. 3,954,726, Fiecchi, May 4, 1976, discloses double salts of S-adenosyl-l-methionine but does not disclose the use of an optically pure diasteriomer of S-adenosyl-l-methionine or defined non-racemic ratios of (S,S)-S-adenosyl-l-methionine to (R,S)-S-adenosyl-l-methionine. U.S. Pat. No. 4,057,686, Fiecchi, Nov. 8, 1977, discloses stable salts of S-adenosyl-l-methionine but does not disclose the use of an optically pure diasteriomer of S-adenosyl-l-methionine or defined non-racemic ratios of (S,S)-S-adenosyl-l-methionine to (R,S)-S-adenosyl-l-methionine. U.S. Pat. No. 4,109,079 Kawahara, et al., Aug. 22, 1978, discloses new stable S-adenosyl-l-methionine salts but does not disclose the use of an optically pure diasteriomer of S-adenosyl-l-methionine or defined non-racemic ratios of (S,S)-S-adenosyl-l-methionine to (R,S)-S-adenosyl-l-methionine. U.S. Pat. No. 4,242,505, Kawahara, et al. Dec. 30, 1980, discloses new stabilizing salts of S-adenosyl-l-methionine but does not disclose the use of an optically pure diasteriomer of S-adenosyl-l-methionine or defined non-racemic ratios of (S,S)-S-adenosyl-l-methionine to (R,S)-S-adenosyl-l-methionine. U.S. Pat. 4,369,177, Kozaki, et al. Jan. 18, 1983, discloses new stable S-adenosyl-l-methionine salts but does not disclose the use of an optically pure diasteriomer of S-adenosyl-l-methionine or defined non-racemic ratios of (S,S)-S-adenosyl-l-methionine to (R,S)-S-adenosyl-l-methionine. U.S. Pat. No. 4,543,408, Gennari, Sep. 24, 1985, discloses new S-adenosyl-l-methionine salts but does not disclose the use of an optically pure diasteriomer of S-adenosyl-l-methionine or defined non-racemic ratios of (S,S)-S-adenosyl-l-methionine to (R,S)-S-adenosyl-l-methionine. U.S. Pat. No. 4,558,122, Gennari, Dec. 10, 1985, discloses new S-adenosyl-l-methionine salts but does not disclose the use of an optically pure diasteriomer of S-adenosyl-l-methionine or defined non-racemic ratios of (S,S)-S-adenosyl-l-methionine to (R,S)-S-adenosyl-l-methionine. U.S. Pat. No. 4,764,603, Zappia, et al. Aug. 16, 1988, discloses the use of new salts of S-adenosyl-l-methionine but does not disclose the use of an optically pure diasteriomer of S-adenosyl-l-methionine or defined non-racemic ratios of (S,S)-S-adenosyl-l-methionine to (R,S)-S-adenosyl-l-methionine. U.S. Pat. No. 4,990,606, Gennari, Feb. 5, 1991, discloses new salts of S-adenosyl-l-methionine but does not disclose the use of an optically pure diasteriomer of S-adenosyl-l-methionine or defined non-racemic ratios of (S,S)-S-adenosyl-l-methionine to (R,S)-S-adenosyl-l-methionine. U.S. Pat. No. 5,073,546, Zappia, et al. Dec. 17, 1991, discloses new salts of S-adenosyl-l-methionine but does not disclose the use of an optically pure diasteriomer of S-adenosyl-l-methionine or defined non-racemic ratios of (S,S)-S-adenosyl-l-methionine to (R,S)-S-adenosyl-l-methionine. U.S. Pat. 5,114,931, Gennari, May 19, 1992, discloses injectable S-adenosyl-l-methionine salts but does not disclose the use of an optically pure diasteriomer of S-adenosyl-l-methionine or defined non-racemic ratios of (S,S)-S-adenosyl-l-methionine to (R,S)-S-adenosyl-l-methionine. U.S. Pat. No. 5,128,249, Gennari, Jul. 7, 1992, discloses new S-adenosyl-l-methionine salts but does not disclose the use of an optically pure diasteriomer of S-adenosyl-l-methionine or defined non-racemic ratios of (S,S)-S-adenosyl-l-methionine to (R,S)-S-adenosyl-l-methionine. U.S. Pat. No. 5,196,402, Braganza, et al. Mar. 23, 1993, discloses the use of S-adenosyl-l-methionine for certain clinical uses but does not disclose the use of an optically pure diasteriomer of S-adenosyl-l-methionine or defined non-racemic ratios of (S,S)-S-adenosyl-l-methionine to (R,S)-S-adenosyl-l-methionine. U.S. Pat. No. 5,466,678, Kawabata, et al. Nov. 14, 1995, discloses the use S-adenosyl-l-methionine to decrease the side effects of chemotherapy but does not disclose the use of an optically pure diasteriomer of S-adenosyl-l-methionine or defined non-racemic ratios of (S,S)-S-adenosyl-l-methionine to (R,S)-S-adenosyl-l-methionine to accomplish this. U.S. Pat. No. 5,137,712, Kask et al, Aug. 11, 1992 discloses the use of S-adenosyl-l-methionine to reverse or prevent side effects of neuroleptic treatment but does not disclose the use of an optically pure diasteriomer of S-adenosyl-l-methionine or defined non-racemic ratios of (S,S)-S-adenosyl-l-methionine to (R,S)-S-adenosyl-l-methionine.

Administration of optically pure diasteriomers of S-adenosyl-l-methionine or defined non-racemic ratios of (S,S)-S-adenosyl-l-methionine to (R,S)-S-adenosyl-l-methionine and their salts of the present invention would have significant utility over a wide range of disorders or conditions associated with low levels of S-adenosyl-l-methionine. Since the two diasteriomeric forms of S-adenosyl-l-methionine of the present invention do not exhibit the same biological activity but rather that the (R,S) S-adenosyl-l-methionine diasteriomer exhibits no biological activity (or even competitive inhibition), it is therefore necessary for a rational pharmaceutical therapy to use the more active diasteriomeric form of S-adenosyl-l-methionine. In this regard, and in view of the (R,S)-S-adenosyl-l-methionine diasteriomer to act as a competitive inhibitor of (S,S,) S-adenosyl-l-methionine in methyltransferase reactions, a more ideal S-adenosyl-l-methionine composition would be the substantially optically pure biologically active (S,S)-S-adenosyl-l-methionine form or a defined non-racemic ratio of (S,S)-S-adenosyl-l-methionine to (R,S)-S-adenosyl-l-methionine to include the highest possible concentration of the (S,S)-S-adenosyl-l-methionine form.

It is an object of the present invention to provide methods for the use of S-adenosyl-l-methionine containing substantially pure biologically active (S,S) S-adenosyl-l-methionine or a defined non-racemic ratio of (S,S)-S-adenosyl-l-methionine to (R,S)-S-adenosyl-l-methionine. It is a further object of the present invention to provide methods of treatment or prevention of conditions that are related to lowered S-adenosyl-l-methionine levels.

Accordingly, there is need in the art for methods related to the use of such substantially optically pure diasteriomeric forms of S-adenosyl-l-methionine and defined non-racemic ratios of (S,S)-S-adenosyl-l-methionine to (R,S)-S-adenosyl-l-methionine diasteriomeric S-adenosyl-l-methionine to increase blood and other tissue and fluid levels of S-adenosyl-l-methionine and to treat conditions which result from low blood and tissue levels of S-adenosyl-l-methionine. The author of this present invention fulfills these needs, and provides further related advantages.

### SUMMARY OF THE INVENTION

Briefly stated, the present invention discloses methods for the use of substantially optically pure diasteriomeric forms of S-adenosyl-l-methionine, defined non-racemic ratios of (S,S)-S-adenosyl-l-methionine to (R,S)-S-adenosyl-l-methionine and their salts. The methods of this present invention have utility in increasing blood and other tissue or fluid levels of S-adenosyl-l-methionine, as well as treating or preventing a wide variety of conditions associated with low blood or other tissue or fluid levels of S-adenosyl-l-methionine and inhibit tumor necrosis factor alpha. Thus in one embodiment, a substantially optically pure diasteriomeric form of S-adenosyl-l-methionine salt or defined non-racemic ratios of (S,S)-S-adenosyl-l-methionine to (R,S)-S-adenosyl-l-methionine and their salts is administered to a warm-blooded animal in need thereof to increase S-adenosyl-l-methionine levels. In another embodiment, a substantially optically pure diasteriomeric form of S-adenosyl-l-methionine salt or defined non-racemic ratios of (S,S)-S-adenosyl-l-methionine to (R,S)-S-adenosyl-l-methionine and their salts is administered to a warm-blooded animal in need thereof to prevent or treat a condition associated with low levels of S-adenosyl-l-methionine. In yet a further embodiment, a substantially optically pure diasteriomeric form of S-adenosyl-l-methionine salt or defined non-racemic ratios of (S,S)-S-adenosyl-l-methionine to (R,S)-S-adenosyl-l-methionine and their salts is administered to a warm blooded animal to prevent and or treat the following conditions: aging, aging of the skin, Alzheimer's disease, rheumatoid arthritis, osteoarthritis, both as an anti-inflammatory as well as to promote new cartilage formation, cancer, conditions of hypomethylation, mitochondrial diseases, hypomethylation of DNA and RNA, nerve damage associated with HIV/AIDS, anxiety, attention deficit disorder and ADHD, sleep regulation, organ preservation for transplant industry, dyslipidemias, excess sebum production, migraines, bile dysfunction caused by pregnancy and use of contraceptive medications, depression, acute and chronic liver disease, cirrhosis of the liver, ischemic reperfusion injury, Parkinson's disease, memory disturbances, memory loss, pancreatitis, intrahepatic cholestasis, inflammation, pain, side effects of administration of chemotherapy, liver disease associated with administration of total parenteral nutrition, liver dysfunction, low tissue levels of glutathione, administration of neuroleptic drugs, administration of cyclosporin A, asthma, and alcohol withdrawal.

### DETAILED DESCRIPTION OF THE INVENTION

As mentioned above, this invention is generally directed to methods of use of a substantially optically pure diasteriomeric form of S-adenosyl-l-methionine salts and to defined non-racemic ratios of (S,S)-S-adenosyl-l-methionine to (R,S)-S-adenosyl-l-methionine and their salts. Such substantially optically pure diasteriomeric forms of S-adenosyl-l-methionine salts or defined non-racemic ratios of (S,S)-S-adenosyl-l-methionine to (R,S)-S-adenosyl-l-methionine and their salts, when administered to a warm blooded animal in need thereof, have utility in the prevention or treatment of conditions associated with low levels of S-adenosyl-l-methionine in warm blooded animals, including humans.

As used herein, the term “conditions” includes diseases, injuries, disorders, indications and/or afflictions that are associated with decreased levels of S-adenosyl-l-methionine. The term “treat” or “treatment” means that the symptoms associated with one or more conditions associated with low levels of S-adenosyl-l-methionine are alleviated or reduced in severity or frequency and the term “prevent” means that subsequent occurrences of such symptoms are avoided or that the frequency between such occurrences is prolonged.

The term “substantially optically pure as used herein, means that the composition contains greater than about 90% of the (S,S)-S-adenosyl-l-methionine diasteriomer by weight in relation to the (R,S) diasteriomer of S-adenosyl-l-methionine, preferably greater than about 94% of the (S,S)-S-adenosyl-l-methionine by weight, and more preferably greater than about 96.999% of (S,S)-S-adenosyl-l-methionine by weight, based upon the total weight of S-adenosyl-l-methionine.

The substantially optically pure diasteriomeric forms of S-adenosyl-l-methionine salts or defined non-racemic ratios of (S,S)-S-adenosyl-l-methionine to (R,S)-S-adenosyl-l-methionine and their salts may be used to prevent and/or treat a variety of conditions associated with lowered levels of S-adenosyl-l-methionine. Due to its ubiquitous distribution in mammalian tissue, S-adenosyl-l-methionine is associated with a variety of conditions: aging, aging of the skin, Alzheimer's disease, rheumatoid arthritis, osteoarthritis, both as an anti-inflammatory as well as to promote new cartilage formation, cancer, conditions of hypomethylation, mitochondrial diseases, hypomethylation of DNA and RNA, HIV/AIDS, anxiety, attention deficit disorder and ADHD, sleep dysregulation, organ preservation for transplant industry, dyslipidemias, excess sebum production, migraines, bile dysfunction caused by pregnancy and use of contraceptive medications, depression, acute and chronic liver disease, cirrhosis of the liver, ischemic reperfusion injury, Parkinson's disease, memory disturbances, memory loss, pancreatitis, intrahepatic cholestasis, inflammation, pain, side effects of administration of chemotherapy, liver disease associated with administration of total parenteral nutrition, liver dysfunction, low tissue levels of glutathione, administration of neuroleptic drugs, administration of cyclosporin A, asthma, and alcohol-withdrawal.

Accordingly, substantially optically pure diasteriomeric forms of S-adenosyl-l-methionine salts or defined non-racemic ratios of (S,S)-S-adenosyl-l-methionine to (R,S)-S-adenosyl-l-methionine and their salts are effective in preventing and/or treating the above conditions due to their ability to increase S-adenosyl-l-methionine levels. To this end, substantially optically pure diasteriomeric forms of S-adenosyl-l-methionine salts or defined non-racemic ratios of (S,S)-S-adenosyl-l-methionine to (R,S)-S-adenosyl-l-methionine and their salts may be used for pharmaceutical, prophylactic and/or cosmetic purposes, and are administered to a warm-blooded animal in an effective amount to achieve a desired result.

In the case of pharmaceutical administration, an effective amount is a quantity sufficient to treat the symptoms of a condition and/or the underlying condition itself. An effective amount in the context of prophylactic administration means an amount sufficient to avoid or delay the onset of a condition and/or its symptoms. Lastly, an effective amount with regard to cosmetic administration is an amount sufficient to achieve the desired cosmetic result.

In a preferred embodiment, substantially optically pure diasteriomeric forms of S-adenosyl-l-methionine salts or a non-racemic mixture of (S,S)-S-adenosyl-l-methionine and (R,S)-S-adenosyl-l-methionine and their salts are administered to a warm-blooded animal as a pharmaceutical, prophylactic or cosmetic composition containing at least one substantially optically pure diasteriomeric form of S-adenosyl-l-methionine salt or a non-racemic mixture of (S,S)-S-adenosyl-l-methionine and (R,S)-S-adenosyl-l-methionine and their salts in combination with at least one pharmaceutically, prophylactically or cosmetically acceptable carrier or diluent. Administration may be accomplished by systemic or topical application, with the preferred mode dependent upon the type and location of the conditions to be treated. Frequency of administration may vary, and is typically accomplished by daily administration. In another embodiment, a pharmaceutical composition of the non-racemic ratio of (S,S)-S-adenosyl-l-methionine to (R,S)-S-adenosyl-l-methionine is preferably from about 80.001% to about 100% of (S,S)-S-adenosyl-l-methionine to about 19.999% to about 0.0% by weight of (R,S)-S-adenosyl-l-methionine.

In yet another embodiment, a pharmaceutical composition of the non-racemic ratio of (S,S)-S-adenosyl-l-methionine to (R,S)-S-adenosyl-l-methionine is more preferably from about 80.001% to about 96.999% of (S,S)-S-adenosyl-l-methionine to about 19.999% to about 3.001% by weight of (R,S)-S-adenosyl-l-methionine.

In yet a further embodiment, a pharmaceutical composition of the non-racemic ratio of (S,S)-S-adenosyl-l-methionine to (R,S)-S-adenosyl-l-methionine is most preferably from about 80.001% to about 95% of (S,S)-S-adenosyl-l-methionine to about 19.999% to about 5% by weight of (R,S)-S-adenosyl-l-methionine.

Systemic administration may be achieved, for example, by injection (e.g., intramuscular, intravenous, subcutaneous or intradermal) or oral delivery of the composition to the warm-blooded animal. Suitable carriers and diluents for injection are known to those skilled in the art, and generally are in the form of an aqueous solution containing appropriate buffers and preservatives. Oral delivery is generally accomplished by formulating the composition in a liquid or solid form, such as a tablet or capsule, by known formulation techniques.

Topical administration may be accomplished, for example, by formulating the composition as solution, cream, gel, ointment, powder, paste, gum or lozenge using techniques known to those skilled in the formulation field. As used herein, topical administration includes delivery of the composition to mucosal tissue of the mouth, nose and throat by, for example, spray or mist application, as well as to the vagina and rectum by, for example, suppository application.

The following example shows how substantially optically pure diasteriomeric forms of S-adenosyl-l-methionine salts or defined non-racemic ratios of (S,S)-S-adenosyl-l-methionine to (R,S)-S-adenosyl-l-methionine and their salts may be used clinically. This example is given to illustrate the present invention, but not by way of limitation. Accordingly, the scope of this invention should be determined not by the embodiment illustrated, but rather by the appended claims and their legal equivalents.

### EXAMPLE 1

(S,S)-S-adenosyl-l-methionine p-toluene sulfonate 400 mg was administered twice daily in an open, non-blind study of 10 volunteers who gave informed consent. All patients had normal results on pre-study medical examinations, including laboratory examinations. Patients received 400 mg of (S,S)-S-adenosyl-l-methionine p-toluene sulfonate in an enteric-coated tablet form twice daily for 14 days or until remission of depression symptoms. The 10 patients satisfied the DSM-III criteria for a major depressive episode. Patients' symptoms were monitored daily using the Hamilton Rating Scale for Depression. 9 patients completed the study. (One patient declined to continue the study after beginning.) Eight of the nine patients who completed the trial improved over the 14 days. One patient had no change at all. No side effects were noted or reported by any of the patients nor as measured by laboratory or physical examination. (S,S)-S-adenosyl-l-methionine p-toluene 400 mg twice daily appeared to be safe and effective in this small, non-blinded study of depression.

I claim:

14 (new) A method to treat or prevent a condition of lowered S-adenosyl-l-methionine tissue and blood levels by increasing S-adenosyl-l-methionine levels in tissue and blood, comprising administering to an animal in need thereof an effective amount of a substantially optically pure (S,S)-S-adenosyl-l-methionine or a pharmaceutically acceptable salt thereof or a defined non-racemic ratio of (S,S)-S-adenosyl-l-methionine to (R,S)-S-adenosyl-l-methionine or pharmaceutically acceptable salts thereof.

15 (new) The method according to claim 14 wherein the (S,S)-S-adenosyl-l-methionine or a pharmaceutically acceptable salt thereof comprises at least 80.001% by weight of the S-adenosyl-l-methionine administered.

16 (new) The method according to claim 14 wherein the (S,S)-S-adenosyl-l-methionine or a pharmaceutically acceptable salt thereof comprises at least 85.00% by weight of the S-adenosyl-l-methionine administered.

17 (new) The method according to claim 14 wherein the (S,S)-S-adenosyl-l-methionine or a pharmaceutically acceptable salt thereof comprises at least 90.00% by weight of the S-adenosyl-l-methionine administered.

18 (new) The method according to claim 14 wherein the (S,S)-S-adenosyl-l-methionine or a pharmaceutically acceptable salt thereof comprises at least 96.999% by weight of the S-adenosyl-l-methionine administered.

19 (new) The method according to claim 14 wherein the condition to be treated is selected from the group consisting of: ageing, ageing of the skin, Alzheimer's disease, osteoarthritis, rheumatoid arthritis, cancer, conditions of hypomethylation, mitochondrial diseases, hypomethylation of DNA and RNA, HIV/AIDS, anxiety, attention deficit disorder and ADHD, sleep dysregulation, organ preservation, dyslipidemias, excess sebum production, migraines, bile dysfunction, bile dysfunction caused by pregnancy and use of contraceptive medications, depression, acute and chronic liver disease, alcohol liver disease, hepatitis B and C, cirrhosis of the liver, ischemic reperfusion injury, strokes, Parkinson's disease, memory disturbances, impaired memory, memory loss, pancreatitis, intrahepatic cholestasis, inflammation, pain, side effects of administration of chemotherapy, total parenteral nutrition induced liver disease, increased levels of tumor necrosis factor alpha, seborrhea, dermatitis, peripheral occlusive arterial disease, low glutathione levels, administration of neuroleptic drugs, administration of cyclosporin A, and asthma.

20 (new) The method of claim 14 wherein the condition to be prevented is selected from the group consisting of: ageing, ageing of the skin, Alzheimer's disease, osteoarthritis, rheumatoid arthritis, cancer, conditions of hypomethylation, mitochondrial diseases, hypomethylation of DNA and RNA, HIV/AIDS, anxiety, attention deficit disorder and ADHD, sleep dysregulation, organ preservation, dyslipidemias, excess sebum production, migraines, bile dysfunction, bile dysfunction caused by pregnancy and use of contraceptive medications, depression, acute and chronic liver disease, alcohol liver disease, hepatitis B and C, cirrhosis of the liver, ischemic reperfusion injury, strokes, Parkinson's disease, MS, memory disturbances, impaired memory, memory loss, pancreatitis, intrahepatic cholestasis, inflammation, pain, side effects of administration of chemotherapy, total parenteral nutrition induced liver disease, increased levels of tumor necrosis factor alpha, seborrhea, dermatitis, peripheral occlusive arterial disease, administration of neuroleptic drugs, administration of cyclosporin A, and asthma.

21 (new) The method of claim 14 wherein the route of administration of a substantially optically pure (S,S)-S-adenosyl-l-methionine or a pharmaceutically acceptable salt thereof or a defined non-racemic ratio of (S,S)-S-adenosyl-l-methionine to (R,S)-S-adenosyl-l-methionine or pharmaceutically acceptable salts thereof is chosen from the group consisting of topical, systemic, oral, intranasal, rectal, and transdermal.

22 (new) A method of claim 14 wherein an effective amount of the substantially optically pure (S,S)-S-adenosyl-l-methionine or a pharmaceutically acceptable salt thereof or a defined non-racemic ratio of (S,S)-S-adenosyl-l-methionine to (R,S)-S-adenosyl-l-methionine or pharmaceutically acceptable salts thereof is administered to a warm-blooded animal to treat a condition of lowered anti-oxidant levels.

23 (new) A method of claim 14 wherein a salt of the substantially optically pure (S,S)-S-adenosyl-l-methionine or of a defined non-racemic ratio of (S,S)-S-adenosyl-l-methionine to (R,S)-S-adenosyl-l-methionine is a member selected from the group consisting of salts of S-adenosyl-l-methionine with hydrochloric acid, sulfuric acid, p-toluenesulfonic acid, and 1,4-butanedisulphonic acid.
